(19) United States
(12) Patent Application Publication
(10) Pub. No.: US 2014/0094645 A1
(43) Pub. Date: Apr. 3, 2014

(54) Methods, Systems, and Devices Relating to a Removable Percutaneous Interface Line

(71) Applicant: Sunshine Heart Company Pty LTD, New South Wales (AU)

(72) Inventors: Daniel Lafontaine, Plymouth, MN (US); William Peters, Remuera (NZ); Martin Cook, Eden Prairie, MN (US); Tolga Tas, Shoreview, MN (US)

(73) Assignee: Sunshine Heart Company Pty LTD, Crows Nest (AU)

(21) Appl. No.: 13/800,654

(22) Filed: Mar. 13, 2013

Related U.S. Application Data
(60) Provisional application No. 61/649,981, filed on May 22, 2012.

(51) Int. Cl. A61M 1/10; A61M 29/00

(52) U.S. Cl. CPC A61M 1/10; A61M 29/00
USPC 600/16

(57) ABSTRACT

The various embodiments disclosed herein relate to percutaneous interface lines and related methods for implanting such lines. Each percutaneous interface line has an internal component coupleable to a percutaneous component with an internal connector. The percutaneous component can have a Y-shaped end having two arms, one arm having a lumen and another arm having at least one wire. The methods for implanting a percutaneous interface line include the use of a tunneling tool having a flexible rod and a replaceable dilator tip.

### CROSS-REFERENCE TO RELATED APPLICATION(S)

This application claims the benefit under 35 U.S.C. § 119(e) to U.S. Provisional Patent Application No. 61/649,981, filed on May 22, 2012, which is hereby incorporated herein by reference in its entirety.

### FIELD OF THE INVENTION

The various embodiments described herein relate generally to a percutaneous interface line (“PIL”) for coupling an implanted medical device (such as, for example, a left ventricular assist device (LVAD), or counter-pulsation or co-pulsation heart assist device) and a controller or driver positioned outside the patient's body, and further to systems having such implanted devices, controllers/drivers, and the percutaneous interface lines that couple those devices.

### BACKGROUND

U.S. Pat. No. 6,132,363 discloses a percutaneous access device (PAD) system that allows both gas and electrical transmission and utilizes an intermediary connector piece that has the patient's own fibroblasts cultured onto the hub of the PAD. This has the proposed advantage of reducing infection. However, its disadvantages include its large size, inflexible nature, and an implantation procedure that requires two or three stages. Specifically, implantation of the PAD device involves making a large skin biopsy, isolating the fibroblasts from the biopsy and growing the cells, then culturing them onto the device (which is a 10 day process). When the culturing process has been completed, the PAD can be implanted in the abdomen, and then the counterpulsation device can be implanted.

Known implantation procedures for percutaneous interface lines (and related devices/technologies) result in large exit site wounds that are necessary to accommodate the tubing or lead termination of the lines. Further, those known procedures typically require the exit site incision to be made from outside the patient's body and further require the line to be inserted into the body through that incision.

There is a need in the art for improved percutaneous interface lines and related systems and methods.

### BRIEF SUMMARY OF THE INVENTION

Discussed herein are various embodiments relating to implantable percutaneous interface lines and related removable connectors.

In Example 1, a percutaneous interface line for an implantable medical device comprises an implantable component, an internal connector, a percutaneous component, and an external connector. The implantable component comprises a first end configured to be sealably coupled to the medical device. The internal connector is configured to be disposed entirely within a patient's body, and further is configured to be sealably coupled to a second end of the implantable component. The percutaneous component comprises a first end configured to be sealably coupleable with the internal connector and a second end. The second end comprises at least one contact ring disposed around a portion of the second end and a lumen defined in the second end. The at least one contact ring is operably coupled to at least one wire, wherein the at least one wire is operably coupled at an opposite end to the implantable component. The external connector is removably coupled to the second end and comprises a connection lumen configured to receive the second end.

Example 2 relates to the percutaneous interface line according to Example 1, wherein the second end can be positioned within the connection lumen at any rotational orientation.

Example 3 relates to the percutaneous interface line according to Example 1, wherein the external connector further comprises at least one threaded hole defined within the connector, wherein the at least one threaded hole is configured to receive a threaded coupling component, wherein the threaded coupling component is configured to physically retain the second end within the external connector.

Example 4 relates to the percutaneous interface line according to Example 3, wherein the at least one threaded hole is perpendicular to the longitudinal axis of the external connector.

Example 5 relates to the percutaneous interface line according to Example 1, wherein the external connector further comprises a locking mechanism configured to physical retain the second end within the external connector.

Example 6 relates to the percutaneous interface line according to Example 1, wherein the percutaneous component comprises a cylindrical body, wherein the at least one wire is operably coupled with the cylindrical body.

Example 7 relates to the percutaneous interface line according to Example 1, wherein the first end of the percutaneous component is a Y-connector.

In Example 8, a percutaneous interface line for an implantable medical device comprises a first component, an internal connector, a second component, and an external connector. The first component comprises a first end configured to be sealably coupled to the medical device and a second end. The internal connector is configured to be disposed entirely within a patient's body and is further configured to be sealably coupled to the second end of the first component. The second component comprises a first end, a line body operably coupled to the first end, and a second end operably coupled to the line body. The first end comprises a first arm and a second arm. The first arm comprises an arm lumen defined in the first arm, the arm lumen in fluid communication with a first arm connector at an end of the first arm, the first arm connector being coupleable to the internal connector. The second arm comprises at least one first end wire electrically coupled to a second arm connector at an end of the second arm. The line body comprises a body lumen in fluid communication with the arm lumen and at least one body wire associated with a wall of the line body, the at least one body wire being electrically coupled to the at least one end wire. The second end comprises at least one ring disposed around a portion of the second end, the at least one ring operably coupled to the at least one body wire, and an end lumen in fluid communication with the body lumen. The external connector is removably coupled to the second end and comprises a connection lumen configured to receive the second end.

Example 9 relates to the percutaneous interface line according to Example 8, wherein the second end can be positioned within the connection lumen at any rotational orientation.

Example 10 relates to the percutaneous interface line according to Example 8, wherein the external connector further comprises at least one threaded hole defined within the connector, wherein the at least one threaded hole is configured to receive a threaded coupling component, wherein the threaded coupling component is configured to physically retain the second end within the external connector.

Example 11 relates to the percutaneous interface line according to Example 10, wherein the at least one threaded hole is perpendicular to the longitudinal axis of the external connector.

Example 12 relates to the percutaneous interface line according to Example 8, wherein the line body is a cylindrical body.

Example 13 relates to the percutaneous interface line according to Example 8, wherein the first end of the second component is a Y-connector.

In Example 14, a method of forming an exit site for a percutaneous interface line comprises providing a tunneling tool and urging the first dilator tip from a location inside a patient's body toward a predetermined location for an exit site. When the first dilator tip is positioned at the predetermined location, the method further comprises urging a guide wire distally through the lumen in the flexible rod and through the opening at the distal end of the first dilator tip until the guide wire pierces the skin, thereby forming the exit site. The method further comprises urging the first dilator tip distally through the exit site, thereby enlarging the exit site, replacing the first dilator tip with a second dilator tip, wherein the second dilator tip has a larger diameter than the first dilator tip, and urging the second dilator tip distally through the exit site, thereby further enlarging the exit site. The tunneling tool comprises a flexible rod comprising a lumen defined through a length of the rod and a replaceable first dilator tip disposed on a distal end of the flexible rod, the first dilator tip comprising a lumen in fluid communication with the lumen in the flexible rod and an opening at a distal end of the dilator tip.

In Example 15, a method of forming an exit site for a percutaneous interface line and implanting the interface line comprises forming an exit site according to the steps of claim 14. Further, the method comprises urging a second component of an interface line distally over the flexible rod until a distal end of the second component is adjacent to or in contact with a proximal portion of the second dilator tip. In addition, the method comprises urging the tunneling tool and second component distally toward and through the exit site until a desired distal length of the second component is positioned externally from the exit site, and urging the tunneling tool distally in relation to the second component, thereby removing the tunneling tool from the patient's body and the second component.

### BRIEF DESCRIPTION OF THE DRAWINGS

FIG. 1A is a schematic view of an implanted percutaneous interface line coupling an external driver to an implanted device, according to one embodiment.

FIG. 1B is a perspective view of a percutaneous (or second) component, according to one embodiment.

FIG. 1C is a cross-sectional view of a percutaneous (or second) component, according to one embodiment.

FIG. 1D is a cross-sectional view of a Y-connector end of a percutaneous (or second) component, according to one embodiment.

FIG. 2A is a side view of a connection that couples a percutaneous interface line to an external driver or controller, according to one embodiment.

FIG. 2B is a side view of a distal end of a percutaneous component of a percutaneous interface line, according to one embodiment.

FIG. 2C is a side view of a distal end of a percutaneous component of a percutaneous interface line positioned in a connection, according to one embodiment.

FIG. 2D is a cross-sectional view of the connection of FIG. 2C.

FIG. 3A is a side view of a anchor, according to one embodiment.

FIG. 3B is a side view of a anchor, according to an alternative embodiment.

FIG. 3C is a side view of a anchor, according to another embodiment.

FIG. 3D is a side view of a anchor, according to a further embodiment.

FIG. 4A is a perspective view of a distal end of a tunneling tool being used to form an exit site, according to one embodiment.

FIG. 4B is a side view of the tunneling tool of FIG. 4A.

### DETAILED DESCRIPTION

The various embodiments disclosed and contemplated herein relate to percutaneous interface lines (“PILs”) (alternatively referred to as “percutaneous drive lines”) and related methods and systems. Each of the various PIL system, device, or method embodiments are intended for use in operably coupling a medical device implanted in a patient (an “implanted device”) with an external control and/or power source (an “external device”). It is understood that “implanted device” includes any device that is implanted or otherwise positioned at least partially within the body of the patient.

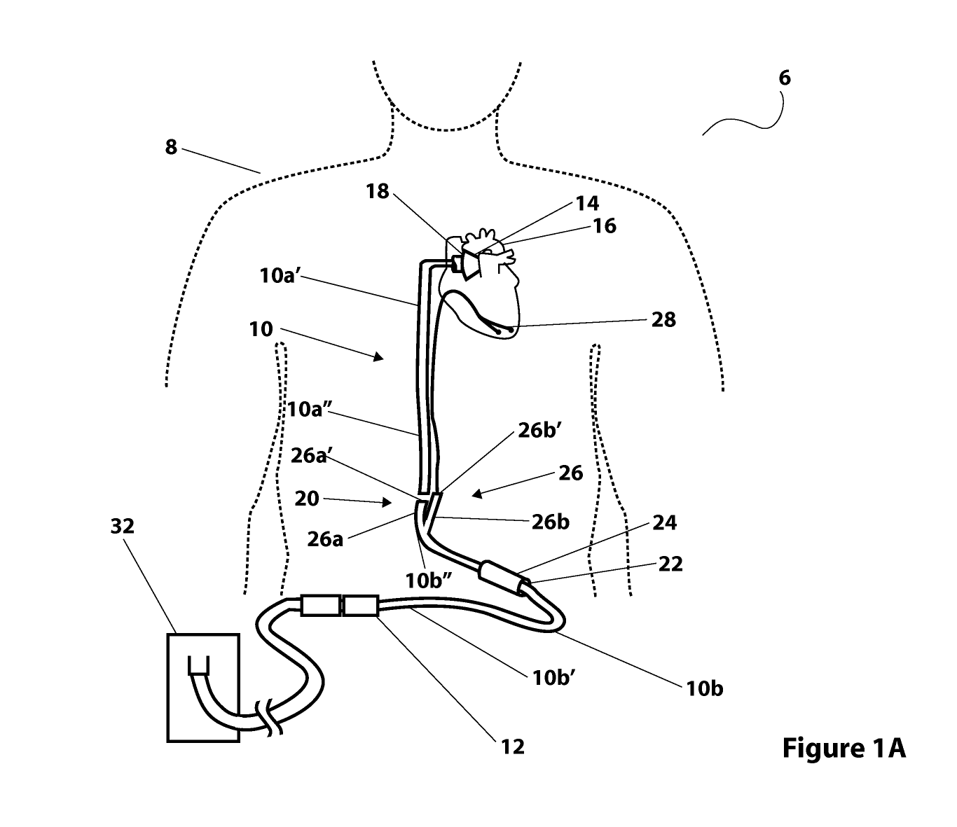

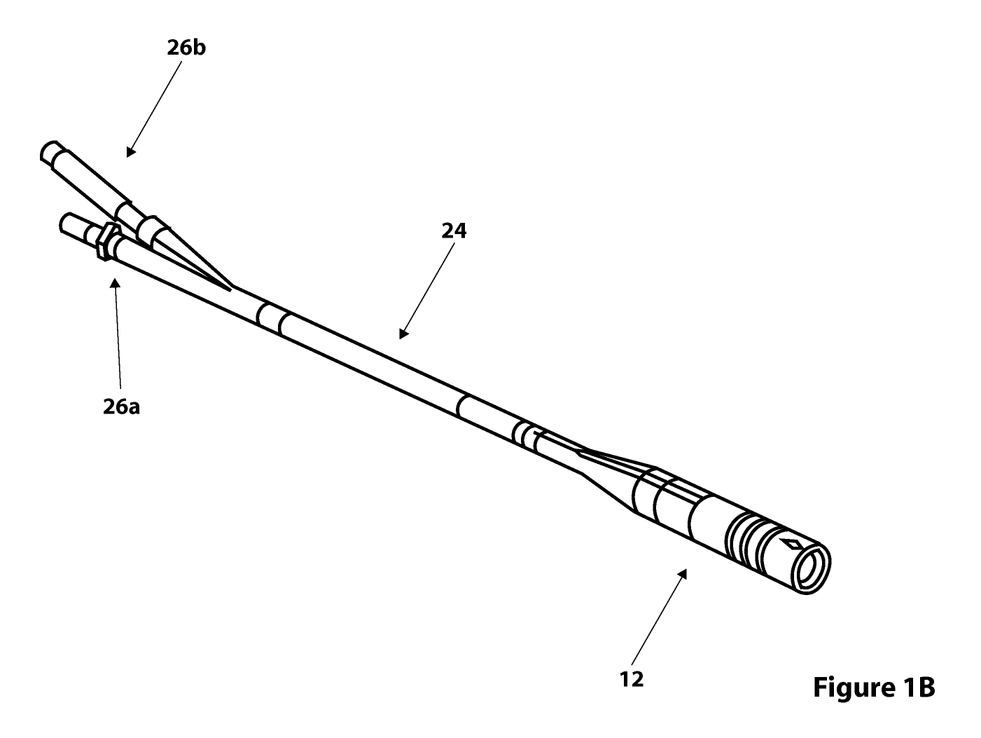

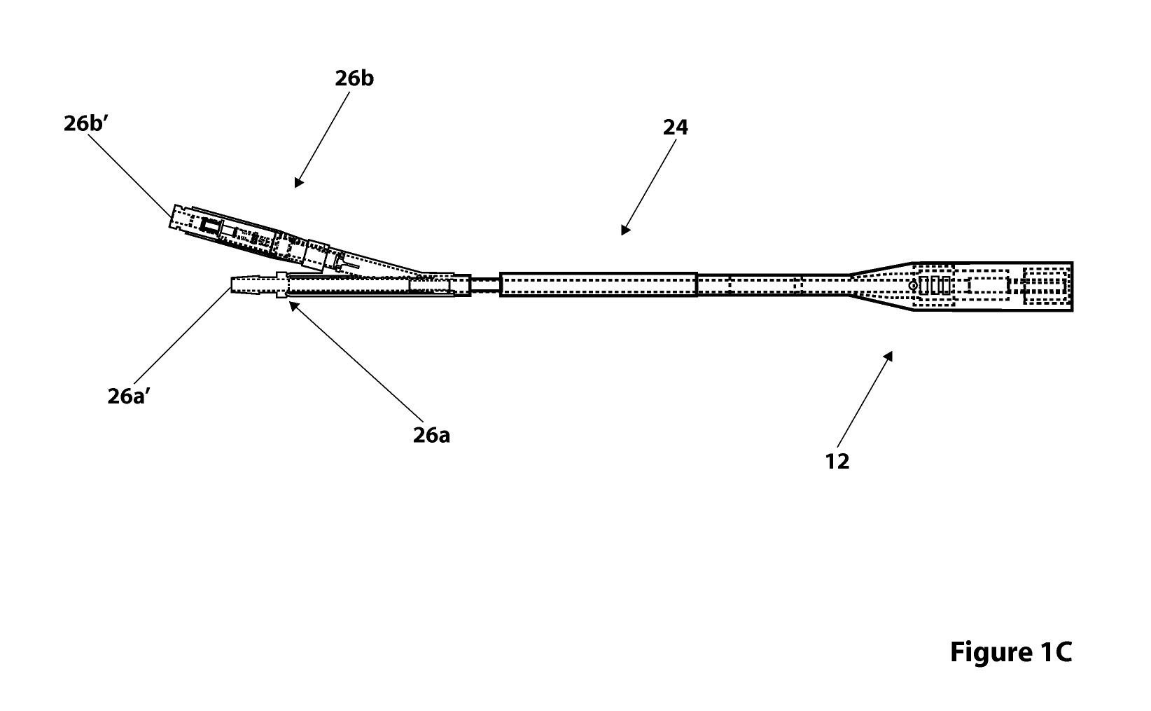

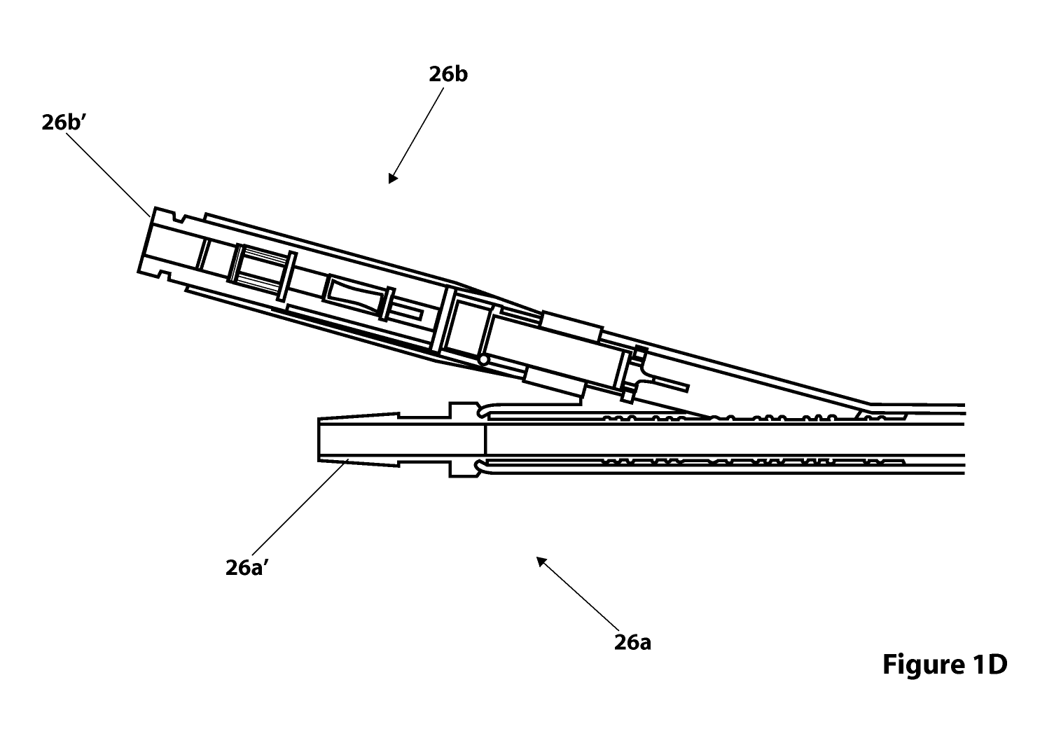

FIG. 1A shows a system 6 according to a first embodiment having a percutaneous interface line (“PIL”) 10 in which the line 10 is implanted in patient 8. The PIL 10 has a first or internal component 10a and a second or percutaneous component 10b (which is shown in further detail in FIGS. 1B, 1C, and 1D), which are removably coupled to each other inside the patient's body via an internal connection 20 that is disposed inside the body of the patient 8. As explained in further detail below, in accordance with one implementation, the PIL 10 is configured to provide both electrical and fluidic coupling of the external device to the implanted device. Alternatively, the PIL 10 can also provide any type of known coupling between an external device and an implanted device and can thereby allow for transfer of any known type of energy or signal, including, but not limited to, signals such as pressure signals, optical sensing signals, or RF communication signals. While the PIL 10 is shown herein with electrical connections, it is understood that there may be electrical components contained within the removeable external connector 12 as well, including resistors, capacitors, inductors, transformers, transistors, or integrated circuits.

As shown in FIG. 1A, the PIL 10 operably connects an external driver 32 to an implanted device 14 such that the driver 32 can power or control the implanted device 14. In this particular embodiment, the system 6 includes an implanted device 14 that is a mechanical heart assist device 14 positioned around the aorta 16 of the patient 8 and having a balloon (not shown), a bushing (not shown), and a wrap 18 to hold the balloon in position around the aorta 16. In addition, the system 6 also includes an ECG lead 28 that is used in conjunction with the mechanical device 14. As such, the driver 32 provides both fluidic and electrical actuation. Alternatively, the PIL 10 can connect the external driver 32 to any implanted medical device or system. In a further embodiment, the external driver 32 can be any external device for controlling or powering a device implanted or otherwise positioned at least partially within a patient's body.

In the implementation depicted in FIG. 1A, the internal component 10a is a tube 10a comprising a lumen (not shown) in fluidic communication with the percutaneous component 10b and the implanted device 14. Alternatively, the internal component 10a can also provide an electrical connection as well.

The internal component 10a of the PIL 10 has a distal end 10a′ sealably connected to the implanted device 14. In this particular embodiment, the distal end 10a′ is coupled to the bushing (not shown) of the heart assist device 14 and is in fluid communication with the balloon (not shown) of the device 14. The internal component 10a of the PIL 10 also has a proximal end 10a″ that is coupled to the connection 20. In one implementation, the internal component 10a is made of a polyurethane-polysiloxane block co-polymer. Alternatively, the internal component 10a can be made of silicone, polyurethane, polyimide, Teflon™, or any similar semi-rigid polymer. In a further alternative, the internal component 10a can be made of any material that can be used in an implanted medical component.

According to one embodiment, the connection 20 is a Luer-lock fitting 20 that provides a fluidic seal at the coupling of the internal component 10a to the percutaneous component 10b. That is, the connection 20 is configured to couple together the internal component 10a and the percutaneous component 10b with a fluidic seal. Alternatively, the connection 20 can be any known connection configured to couple together two components of a PIL such that the connection provides a fluid seal, such that any fluid—including any gas or liquid—is sealably retained within the PIL when the two components 10a, 10b are coupled to the connection 20.

The percutaneous component 10b of the PIL 10 is shown positioned percutaneously through an exit site 22. The percutaneous component 10b has a distal end 10b″, which is positioned inside the patient 6 and connected to the connection 20. As best shown in FIGS. 1A, 1B, 1C, and 1D, the distal end 10b″ in this embodiment is a “Y-connector” 26, which is a Y-shaped component in which two different arms 26a, 26b extend from the single lumen at the distal end 10b″ of the percutaneous component 10b, with a connector 26a′, 26b′ at the end of the arms 26a, 26b, respectively.

Please note that the length of this embodiment as shown in FIGS. 1A-1D, along with the other embodiments disclosed and depicted herein, is merely exemplary and provided mainly because that particular length is easy to depict in the figures. This exemplary length as shown does not limit the actual length of the percutaneous component 1b embodiments as disclosed and contemplated herein.

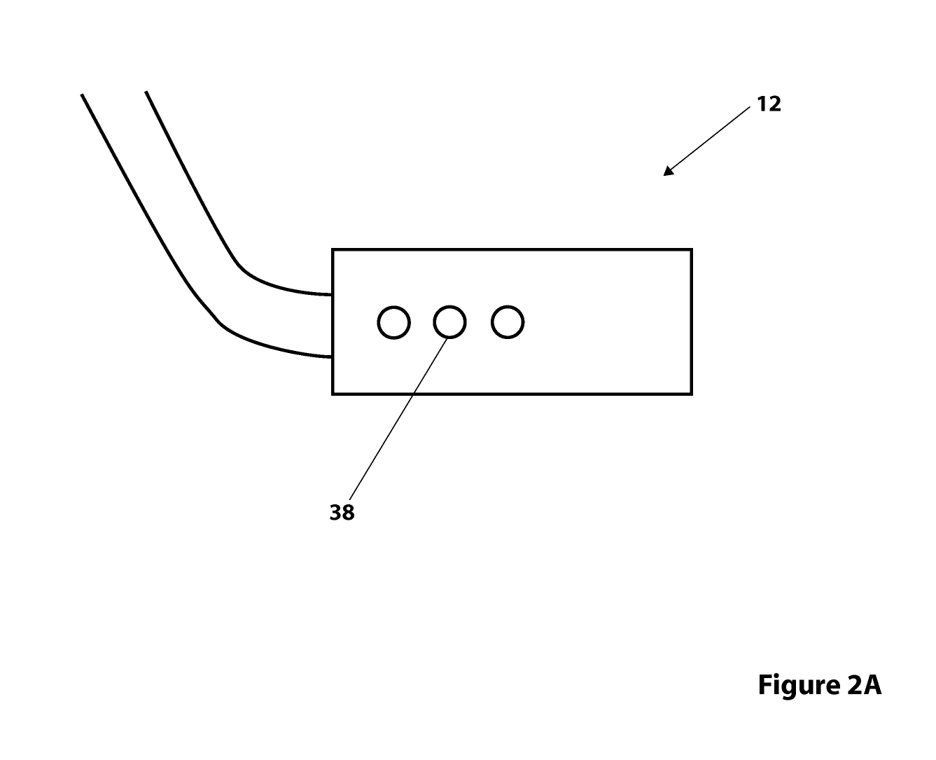

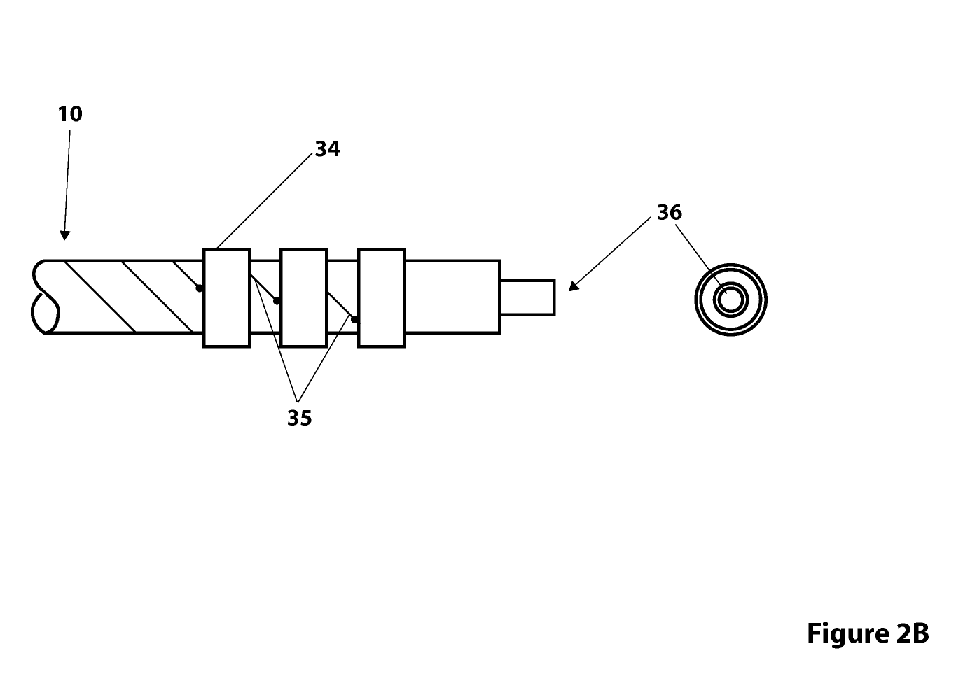

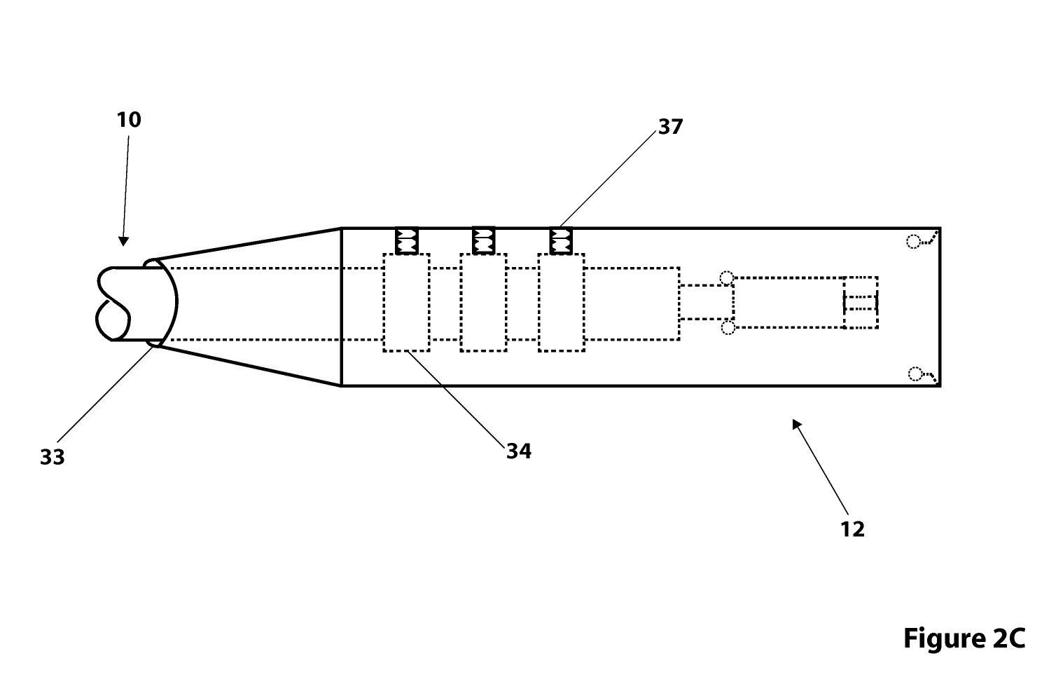

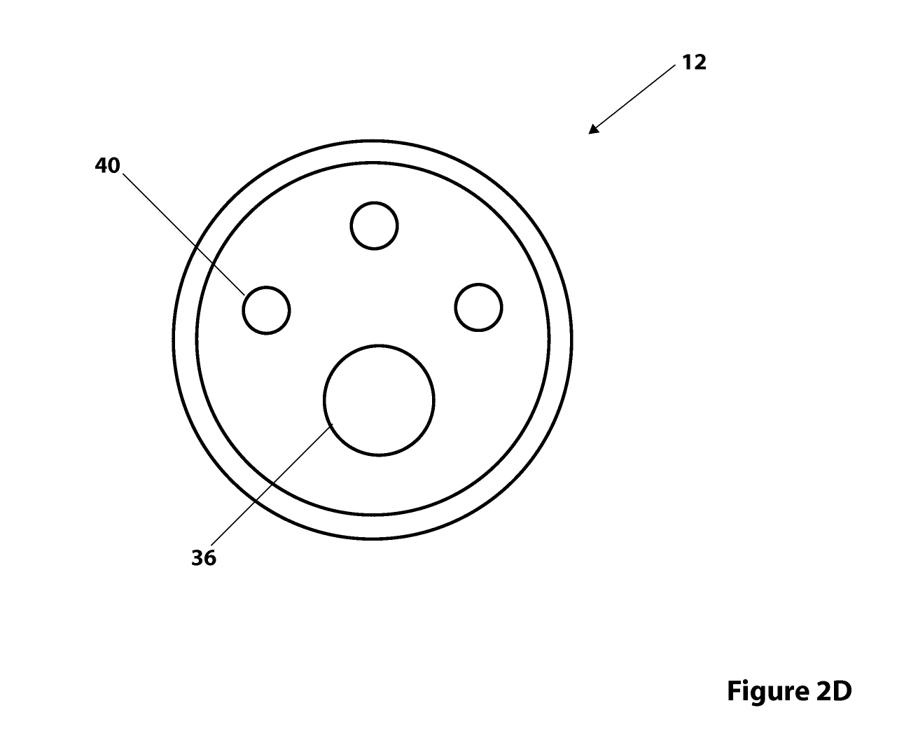

In the embodiment of FIGS. 1A-1D, the first arm 26a of the Y-connector 26 has a lumen (not shown) configured to allow fluid, such as, for example, gas, to pass through the arm 26a. The first arm 26a is coupleable to the connection 20 at connector 26a′ such that the lumen in the first arm 26a is in fluidic communication with the lumen (not shown) in the internal component 10a via the connection 20. Further, the second arm 26b of the Y-connector 26 is an electrical arm 26b that has at least one wire 35 (as best shown in FIG. 2B) positioned in the arm 26b. The at least one wire 35 (as best shown in the embodiment depicted in FIG. 2B) is electrically coupled with the connector 26b′. According to this implementation, the connector 26b′ is electrically coupled to the ECG lead 28, which extends from the connector 26b′ and is positioned on, around, or near the patient's heart as shown.

The proximal end 10b′ of the percutaneous component 10b is positioned outside of the patient and is connected to the external driver 32 at external connection 12. According to one embodiment, the connection 12 is a connection that provides both a fluidic seal and an electrical connection and allows the proximal end 10b′ of the component 10b to be removable from the connection 12.

One embodiment of a connection 12 is depicted in FIGS. 2A, 2B, 2C, and 2D. The connection 12 is also referred to as a connector 12, a removable connector 12, or an external connector 12 (or some variation thereof). As best shown in FIG. 2C, the connection 12 is a cylindrical component that defines a lumen 33 configured to receive the distal end 10b′ of the percutaneous component 10b. The connection 12 also has three threaded holes 37 defined in the connection 12 (as shown in FIG. 2C) and configured to receive threaded set screws 38 (as shown in FIG. 2A) that are configured to retain the distal end 10b′ within the connection 12. More specifically, when the distal end 10b′ has been inserted into the lumen 33, the set screws 38 can be positioned in the holes 37 such that the screws 38 contact the distal end 10b′, thereby providing frictional or physical resistance to the removal of the distal end 10b′ from the lumen 33. Alternatively, the connection 12 can have at least one threaded hole. In a further alternative, the connection 12 has a locking mechanism such as a snap lock or snap fit locking mechanism that can be used to retain the distal end 10b′ of the percutaneous component 10b within the lumen 33 of the connection 12. In yet another alternative, any other known mechanism for retaining the distal end 10b′ within the lumen 33 can be used.

According to various alternative implementations, it is understood that the connection 12 can take any form or use any components to electrically connect the percutaneous component 10b and the external driver 32, including, for example, resistors, capacitors, inductors, transformers, transistors, or integrated circuits.

As best shown in FIG. 2B, one embodiment of the distal end 10b′ of the percutaneous component 10b has three connector rings 34 disposed around the component 10b. Each of the rings 34 is electrically coupled to at least one wire 35, wherein each wire 35 extends from the rings 34 along the length of the percutaneous component 10b and is positioned in the second arm 26b as discussed above. In one embodiment, each wire 35 is incorporated into the tube of the percutaneous component 10b in a spiral configuration such that each wire is “wound around” the percutaneous component 10b. Each wire 35 can be positioned or embedded or otherwise placed within the wall of the component 10b. Alternatively, the wires 35 can be positioned outside or inside of the wall. In yet another embodiment, the wires 35 can extend along the length of the component 10b in a non-spiral configuration.

The connection 12 is configured to provide both a fluid connection and an electrical connection between the percutaneous component 10b and the driver 32, thereby connecting the driver 32 to the implanted device 14 both fluidically (or pneumatically) and electrically. That is, in the specific embodiment as best depicted in FIGS. 2C and 2D, the proximal end of the connection 12 as shown in FIG. 2D has a lumen 36 that is in fluidic communication with the lumen (not shown) in the percutaneous component 10b. Further, the proximal end also has three electrical connector pins 40 that are in electrical communication with the connector rings 34 when the end 10b′ of the percutaneous component 10b is positioned correctly within the connection 12. As such, the lumen 36 and the connector pins 40 provide for fluidic and electrical coupling, respectively, to the driver 32. Alternatively, the PIL 10 can have fewer or more fluidic and/or electrical connections, thereby resulting in fewer or more therapeutic use options. For example, the PIL 10 can have four or more wires or other types of electrical components. In another example, the PIL 10 can have two or more fluidic lumens that can be used to drive more than one implanted device or more than one component of the same device.

According to one embodiment, the connector 12 is removable. That is, the connector 12 is removably coupled to the proximal end of the percutaneous component 10b such that the connector 12 can be easily coupled to and uncoupled from the component 10b. The removability of the connector 12 makes it possible for the percutaneous component 10b to be surgically positioned as described herein before being attached to the connector 12, thereby providing the patient with easier access to the connector 12. In contrast, the known devices in the art do not have a detachable external connector and therefore such a connector cannot be positioned by the patient for ease of use without unwanted twisting, kinking, and other unwanted damage to the percutaneous line over time. Further, in devices having a non-detachable external connector, any damage to the external portions of the percutaneous line or other external components renders the entire line and all external components unusable and makes it necessary to do a full replacement of the entire line, which requires surgery. In addition, for any patient who gains or loses weight over time, the lack of a detachable external connector means that the patient cannot get a more suitable connector position or external length without replacing the entire line, thereby limiting the patient's ability to operate the external connector and line in the same manner as when the patient originally received the device.

As such, various connector 12 embodiments described and contemplated herein allow for (1) replaceability of the connector 12 and the percutaneous component 10b, (2) percutaneous delivery of the percutaneous component 10b, (3) a larger connector 12 in comparison to the prior art connectors positioned within the patient, thereby resulting in easier handling for the patient and the medical professionals, and (4) installation in any orientation, thereby ensuring flexibility for patients during connection and disconnection of the connector 12 and the percutaneous component 10b. The proximal end of the percutaneous component 10b can be positioned within the lumen 36 of the connector 12 in any rotational orientation and the couplings (electrical, fluidic, and any other couplings) are operable regardless of orientation.

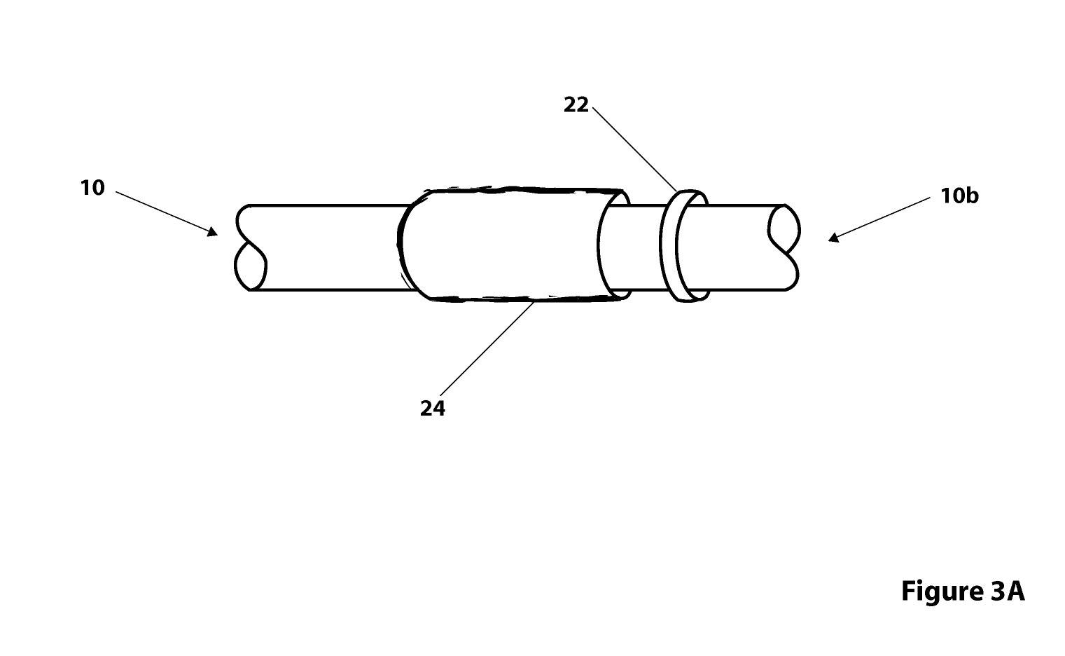

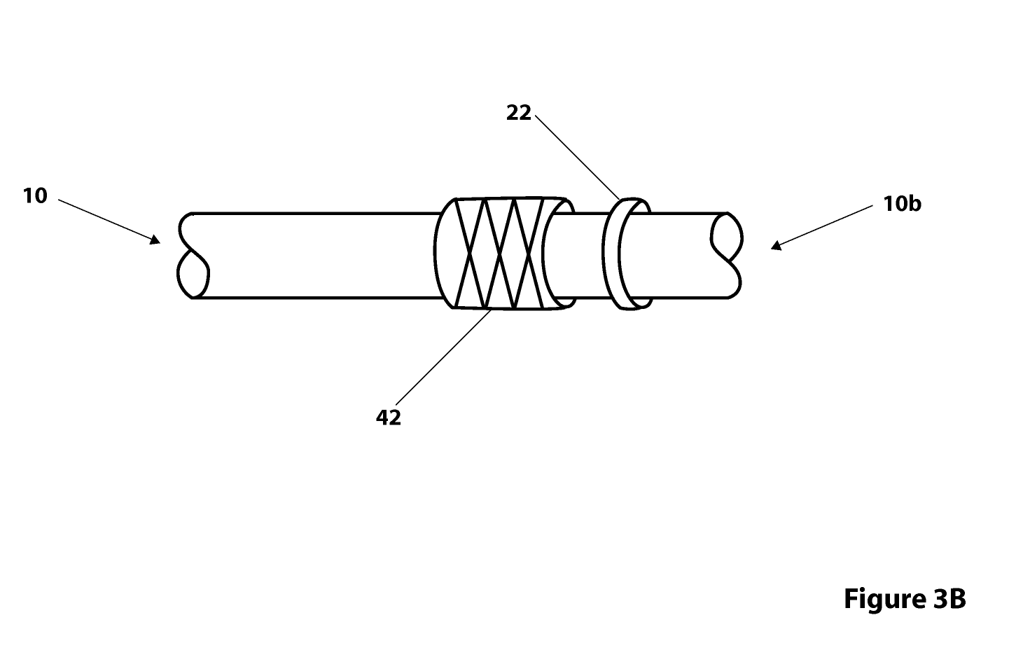

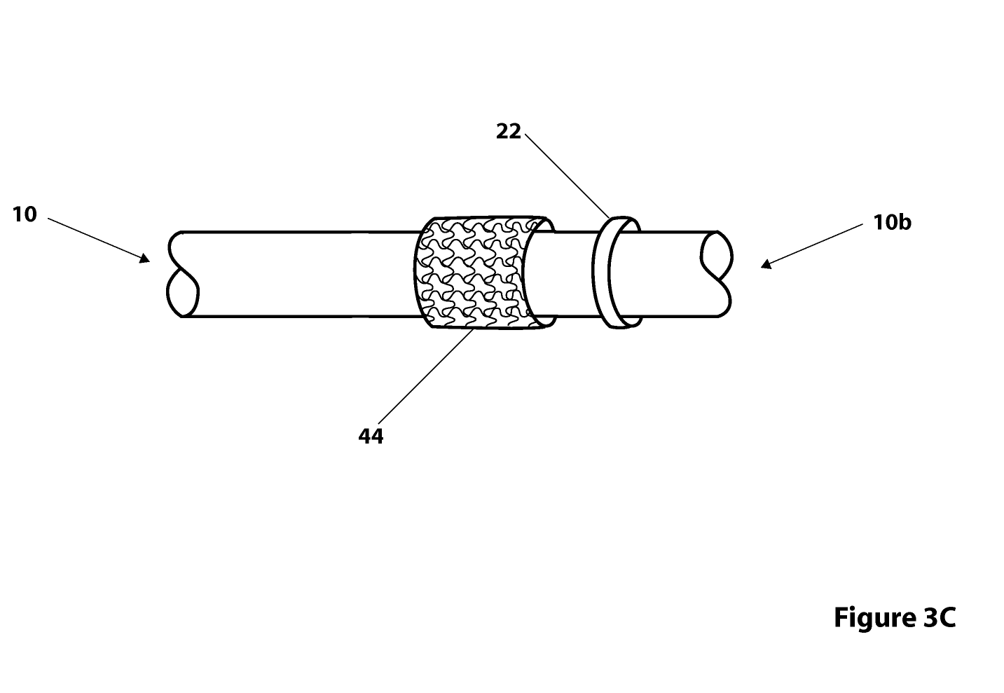

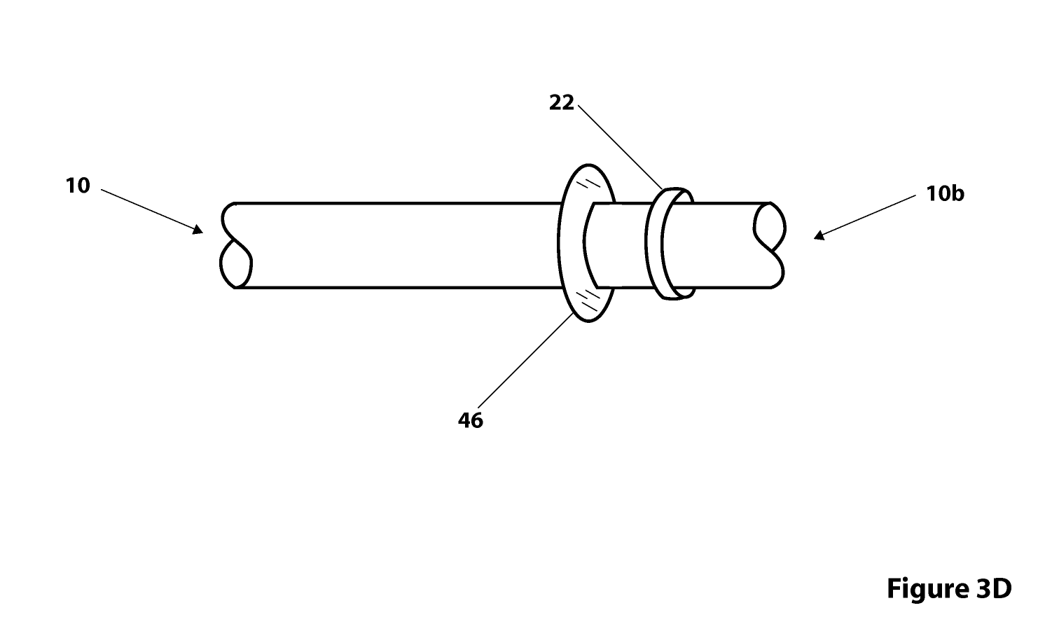

As best shown in FIGS. 1A, 1B, 1C, and 3A, in accordance with one implementation, the implanted portion of the percutaneous component 10b has a collar or anchor 24 coupled to the component 10b that helps to anchor the PIL 10 within the patient. One specific example of an anchor 24 is a polyester collar 24 as shown in FIG. 3A. An alternative embodiment of an anchor 42 that is an expandable stent 42 is depicted in FIG. 3B. The expandable stent 42 is configured to help anchor the PIL 10 within the patient by expanding in a known fashion, thereby attaching to the tissue surrounding the anchor 42.

In a further alternative as shown in FIG. 3C, the anchor 44 is a flocking 44, which, for purposes of this application, is an anchor 44 having a diameter that is larger than the percutaneous component 10b. More specifically, in one embodiment, the flocking 44 has a diameter of at least about 2 to 10 times the diameter of the tubing. A flocking 44 can be made of polyester or any other known fibrous fabric used for prosthetic tissue attachment.

According to certain embodiments, any version of the anchors 24, 42, or 44 has a length ranging from about 10 mm to about 100 mm. Alternatively, the anchor 24, 42, or 44 has a length that extends along the entire, or substantially the entire, implantable portion of 10b.

In yet another alternative, the anchor 46 is a donut- or disk-shaped anchor 46 positioned around the percutaneous component 10b, as depicted in FIG. 3D. In certain embodiments, the donut- or disk-shaped anchor 46 has a diameter that is equal to or greater than the length of the anchor 46. In one implementation, the anchor 46 is made of a plastic material, such as, for example, polycarbonate or Bionate.

According to one embodiment, the anchor 24, 42, 44, or 46 is positioned along the percutaneous component 10b such that it is spaced proximally from the distal end 10b″ of the component. According to one specific implementation, the anchor 24, 42, 44, or 46 is positioned along the percutaneous component 10b and the percutaneous component 10b is positioned within the patient such that the anchor 24, 42, 44, or 46 is positioned internally about 20 to 50 mm from the exit site 22. In a further embodiment, the anchor 24, 42, 44, or 46 is configured to be positioned below the subcutaneous muscle layer when it is implanted. Alternatively, the anchor 24, 42, 44, or 46 is configured to be positioned under or beneath the skin at the exit site 22 and substantially adjacent thereto.

The percutaneous component 10b, in accordance with one implementation, can be made of a different material than the internal component 10a. That is, the percutaneous component 10b can be made of silicone or silicone-polyurethane co-polymer. In addition, in certain implementations, the percutaneous component 10b is more flexible than the internal component 10a. In yet another embodiment, the percutaneous component 10b can be wire-wound. That is, the component 10b can be made with a wire skeleton—or other metal structure—within a polymeric material. One such configuration is a spiral wire configuration. Alternatively, any known metal structure can be used.

In use, one embodiment of the driver or controller 32 as described herein is a counterpulsation pump that uses the three electrical connections to sense the depolarization of the heart directly and create a signal to actuate the heart assist device 14 that is timed according to ventricular depolarization, or systole. A positive sense lead and a negative sense lead (such as the leads 28 as shown in FIG. 1A) detect the ECG signal and the third lead is a right leg drive signal used to cancel noise from the sensed signals. At the start of depolarization, the heart begins to contract the ventricle in systole and the controller 32 is configured to deflate the implanted balloon device 14 (as shown in FIG. 1A) to aid systolic ejection from the ventricle. After a programmed delay based on heart rate or a sensed delay based on the closure of the aortic valve, the controller 32 is configured to inflate the device 14 to provide an additional cardiac output from the aorta to the heart in diastole and the rest of the body and organs. Alternatively, the controller 32 can be any known controller or driver 32 configured to control or power any implanted device 14.

The percutaneous interface line 10 described above can be implanted into a patient in the following fashion.

According to one embodiment, the internal component 10a is first implanted in the patient. The component 10a can be implanted with the medical device (such as, for example, the mechanical heart assist device 14 in FIG. 1A and discussed above). Alternatively, the medical device can be implanted first, and then the component 10a can be implanted and coupled to the medical device. The internal component 10a can be implanted using any known methods or procedures.

When the internal component 10a has been implanted (or when a previously implanted percutaneous component 10b is to be removed and replaced), the (new) percutaneous component 10b can be implanted. Unlike prior methods in which the exit site 22 is formed first and then access is created from the exit site 22 to the connection 20 at the proximal end 10a″ of the internal component 10a, the instant embodiment relates to forming access (or “tunneling”) from the connection 20 to the desired location for the exit site 22 and forming the exit site 22 after the access or tunneling has occurred.

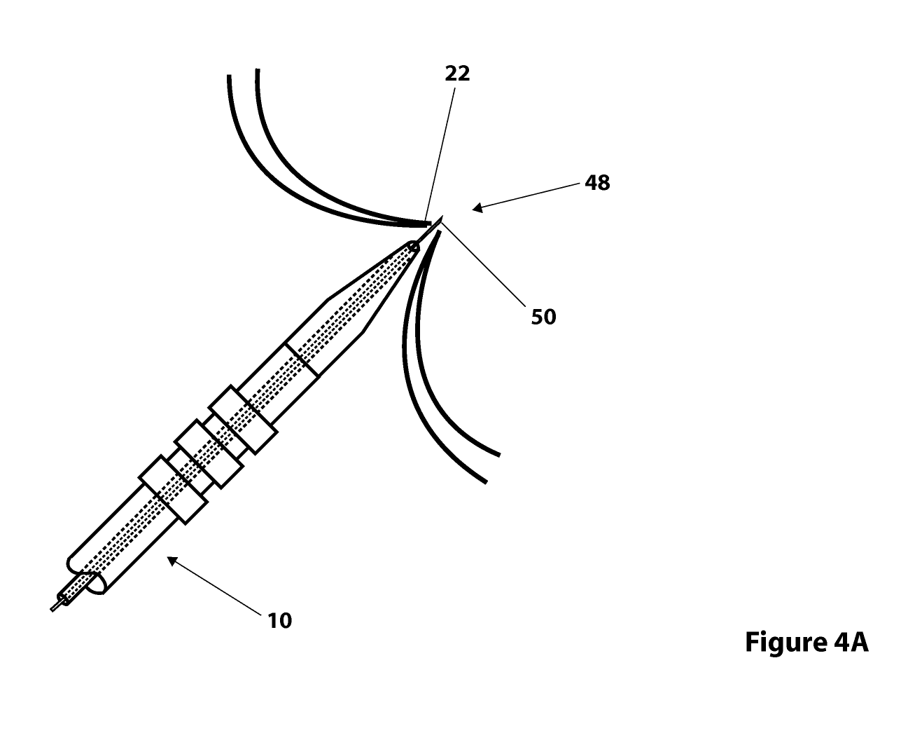

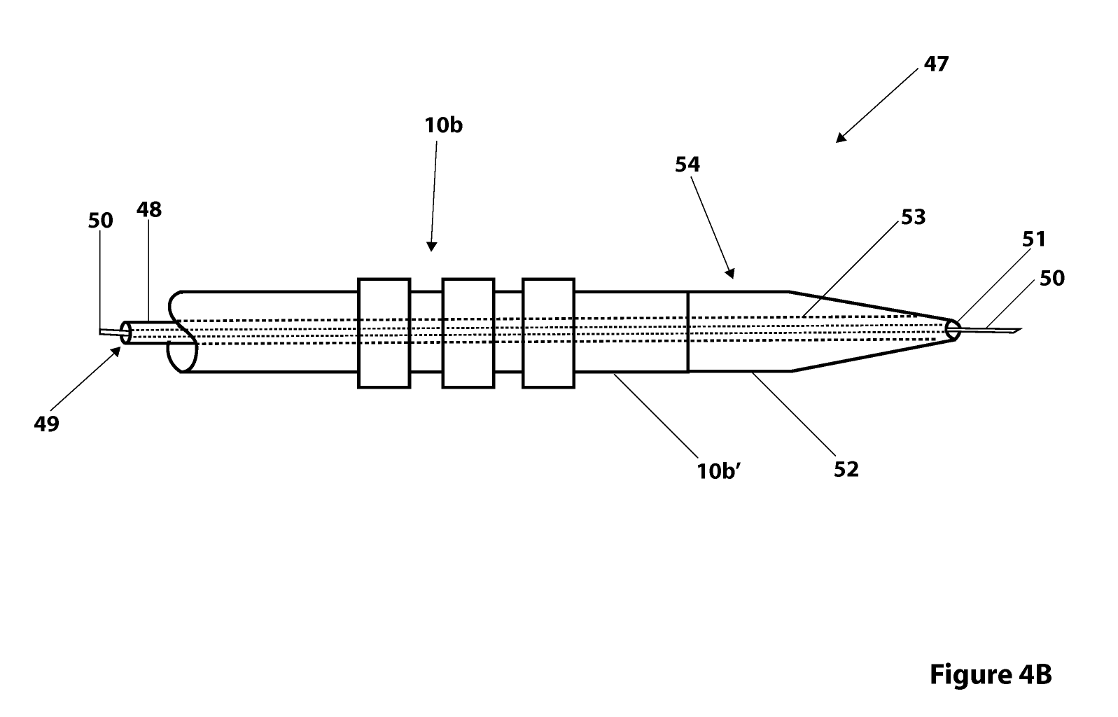

FIGS. 4A and 4B depict a tunneling tool 47 according to one embodiment that can be used to perform the tunneling or access procedure. As best shown in FIG. 4B, the tool 47 has a flexible rod 48 having a dilator tip 52 on the distal end of the rod 48. The rod 48 has a lumen 49 running along the length of the rod 48. In addition, the dilator tip 52 has a lumen 53 formed through the middle of the tip 52 with an opening 51 to the lumen 53 at the distal end of the tip 52. The lumen 49 in the rod 48 is in communication with the lumen 53 in the tip 52 such that the lumens 49, 53 are configured to receive or be positioned over a guide wire 50. In addition, the rod 48 and the tip 52 are configured such that the proximal end 10b′ of the percutaneous component 10b can be positioned over the rod 48 as shown in FIG. 4B and positioned adjacent to or against the tip 52. That is, the rod 48 is configured to be positionable within the lumen (not shown) in the percutaneous component 10b such that the percutaneous component 10b can be positioned over the rod 48 and the proximal end 10b′ can be positioned against the dilator tip 52. According to one implementation, the proximal end 10b′ and the dilator tip 52 have substantially the same diameter.

In use, the tunneling tool 47 can be used in the following manner. Beginning at the site of the connection 20 (the proximal end 10a″ of the internal component 10a), the dilator tip 52 of the tool 47, is urged toward the desired exit site at the skin of the patient. The location of the desired exit site is previously determined by the doctor and/or the patient, and is typically positioned vertically somewhere between the patient's lowest rib and the patient's beltline and horizontally between the midline and either side of the patient's abdomen. When the dilator tip 52 reaches the skin at the desired location for the exit site, the tip 52 is urged against the skin, thereby creating a “tenting” action. That is, the tip 52 is urged against the skin without breaking through the skin such that the portion of the skin in direct contact with the tip 52 is urged away from the patient's body, thereby creating a tent-like configuration. This allows the surgeon (or other user) to identify the exact location of the tip 52 on the skin and thereby allows the user to adjust the location of the tip 52 along the skin if necessary/desired.

When the tip 52 is positioned as necessary/desired at the appropriate location along the skin, the guide wire 50 is positioned through the lumens 49, 53 in the tool 47 and urged distally out of the hole 51 at the dilator tip 52 in a fashion similar to that shown in FIG. 4B. According to one embodiment, the guide wire 50 has a needle-like tip at its distal end such that the urging of the guide wire 50 in a distal direction urges the needle-like tip through the skin, thereby piercing the skin such that the tip exits out of the skin as shown in FIG. 4A, thereby creating the exit site 22.

Once the initial piercing of the skin has been accomplished, the tunneling tool 47 is used to progressively enlarge the size of the exit site 22. That is, the tunneling tool 47 is first urged distally such that the dilator tip 52 is urged through the exit site 22, thereby dilating or enlarging the site 22. In one embodiment, the dilator tip 52 has a size ranging from about 3 French to about 5 French.

Once the initial dilator tip 52 has been used to enlarge the site 22, a bigger dilator is used. That is, the tool 47 is removed from the exit site 22 or otherwise moved proximally over the guidewire (with the guidewire remaining in place) to move the dilator tip 52 away from the exit site 22, and then the dilator tip 52 is removed and replaced with a larger dilator tip 52. In one embodiment, the larger dilator tip 52 has a size ranging from about 12 French to about 20 French. The larger tip 52 is then urged over the guidewire and through the exit site 22, thereby further enlarging the site 22.

Subsequently, in one implementation, another, even larger dilator tip 52 replaces the previous tip 52 and used to further enlarge the site 22 using steps similar to those described above. It is understood that any known number of dilator tips of any known sizes can be used, so long as each is replaced with a larger tip until the desired size of the exit site 22 is achieved.

When the desired size for the exit site 22 has been achieved, the tool 47 is again urged proximally away from the exit site 22. At this point, the proximal end 10b′ of the percutaneous component 10b is positioned over the rod 48 and positioned adjacent to or against the tip 52 as shown in FIG. 4B. The tool 47 is then urged distally through the exit site 22 as shown in FIG. 4A such that the percutaneous component 10b is urged through the site 22 as well. The tool 47 is moved distally until the appropriate length of the percutaneous component 10b is positioned external to the patient's body. In one embodiment, the length of component 10b to be positioned outside the body is determined by marking an external portion of the component 10b such that when that mark is visible, the component 10b is deemed to be positioned appropriately and the distal urging is stopped.

When the percutaneous component 10b is positioned correctly/as desired, the tool 47 is removed by uncoupling the tool 47 from the component 10b and urging the tool 47 and guidewire 50 distally such that the rod 48 is urged distally out of the proximal end 10b′ of the component 10b. When the tool 47 has been removed, the connection 12 can be coupled to the component 10b.

In accordance with one embodiment, the process of performing the various steps described herein from inside the patient's body can help to minimize risk of infection and dermal injury. That is, the forming of the exit site 22, the enlarging of the exit site 22, and the positioning of the percutaneous component 10b all starting from inside the body can reduce infection and injury in comparison to the standard procedures that start from outside the body. More specifically, without being limited by theory, the above steps are performed from inside the body such that the dermal layers are positioned in a tenting manner during each step, thereby maintaining the elastin and collagen layers in an orientation or configuration that prevents or minimizes the entry of germs or debris into the exit site 22 while minimizing the dermal injury caused during formation and enlargement of the site 22.

It will be appreciated by the persons skilled in the art that numerous variations and/or modifications can be made to the embodiments disclosed herein without departing from the spirit or scope of the invention as broadly defined.

What is claimed is:

1. A percutaneous interface line for an implantable medical device, the interface line comprising:
(a) an implantable component comprising a first end configured to be sealably coupled to the medical device;
(b) an internal connector configured to be disposed entirely within a patient's body, wherein the internal connector is configured to be sealably coupled to a second end of the implantable component; and
(c) a percutaneous component comprising:
(i) a first end configured to be sealably coupleable with the internal connector;
(ii) a second end comprising:
(A) at least one contact ring disposed around a portion of the second end, the at least one contact ring being operably coupled to at least one wire, wherein the at least one wire is operably coupled at an opposite end to the implantable component;
(B) a lumen defined in the second end; and
(d) an external connector removably coupled to the second end, the external connector comprising a connection lumen configured to receive the second end.

2. The percutaneous interface line of claim 1, wherein the second end can be positioned within the connection lumen at any rotational orientation.

3. The percutaneous interface line of claim 1, wherein the external connector further comprises at least one threaded hole defined within the connector, wherein the at least one threaded hole is configured to receive a threaded coupling component, wherein the threaded coupling component is configured to physically retain the second end within the external connector.

4. The percutaneous interface line of claim 3, wherein the at least one threaded hole is perpendicular to the longitudinal axis of the external connector.

5. The percutaneous interface line of claim 1, wherein the external connector further comprises a locking mechanism configured to physical retain the second end within the external connector.

6. The percutaneous interface line of claim 1, wherein the percutaneous component comprises a cylindrical body, wherein the at least one wire is operably coupled with the cylindrical body.

7. The percutaneous interface line of claim 1, wherein the first end of the percutaneous component is a Y-connector.

8. A percutaneous interface line for an implantable medical device, the interface line comprising:
(a) a first component comprising a first end configured to be sealably coupled to the medical device and a second end;
(b) an internal connector configured to be disposed entirely within a patient's body, wherein the internal connector is configured to be sealably coupled to the second end of the first component;
(c) a second component comprising:
(i) a first end comprising:
(A) a first arm comprising an arm lumen defined in the first arm, the arm lumen in fluid communication with a first arm connector at an end of the first arm, the first arm connector being coupleable to the internal connector; and
(B) a second arm comprising at least one first end wire electrically coupled to a second arm connector at an end of the second arm;
(ii) a line body operably coupled to the first end, the line body comprising
(A) a body lumen in fluid communication with the arm lumen; and
(B) at least one body wire associated with a wall of the line body, the at least one body wire being electrically coupled to the at least one end wire; and
(iii) a second end operably coupled to the line body, the second end comprising
(A) at least one ring disposed around a portion of the second end, the at least one ring operably coupled to the at least one body wire; and
(B) an end lumen in fluid communication with the body lumen; and
(d) an external connector removably coupled to the second end, the external connector comprising a connection lumen configured to receive the second end.

9. The percutaneous interface line of claim 8, wherein the second end can be positioned within the connection lumen at any rotational orientation.

10. The percutaneous interface line of claim 8, wherein the external connector further comprises at least one threaded hole defined within the connector, wherein the at least one threaded hole is configured to receive a threaded coupling component, wherein the threaded coupling component is configured to physically retain the second end within the external connector.

11. The percutaneous interface line of claim 10, wherein the at least one threaded hole is perpendicular to the longitudinal axis of the external connector.

12. The percutaneous interface line of claim 8, wherein the line body is a cylindrical body.

13. The percutaneous interface line of claim 8, wherein the first end of the second component is a Y-connector.

14. A method of forming an exit site for a percutaneous interface line, the method comprising:
providing a tunneling tool comprising:
(a) a flexible rod comprising a lumen defined through a length of the rod;
(b) a replaceable first dilator tip disposed on a distal end of the flexible rod, the first dilator tip comprising a lumen in fluid communication with the lumen in the flexible rod and an opening at a distal end of the dilator tip;
urging the first dilator tip from a location inside a patient's body toward a predetermined location for an exit site;
when the first dilator tip is positioned at the predetermined location, urging a guide wire distally through the lumen in the flexible rod and through the opening at the distal end of the first dilator tip until the guide wire pierces the skin, thereby forming the exit site;
urging the first dilator tip distally through the exit site, thereby enlarging the exit site;
replacing the first dilator tip with a second dilator tip, wherein the second dilator tip has a larger diameter than the first dilator tip; and
urging the second dilator tip distally through the exit site, thereby further enlarging the exit site.

15. A method of forming an exit site for a percutaneous interface line and implanting the interface line, the method comprising:
forming an exit site according to the steps of claim 14;
urging a second component of an interface line distally over the flexible rod until a distal end of the second component is adjacent to or in contact with a proximal portion of the second dilator tip;
urging the tunneling tool and second component distally toward and through the exit site until a desired distal length of the second component is positioned externally from the exit site; and
urging the tunneling tool distally in relation to the second component, thereby removing the tunneling tool from the patient's body and the second component.
